(19) United States
(12) Patent Application Publication
(10) Pub. No.: US 2010/0248239 A1
(43) Pub. Date: Sep. 30, 2010

(54) METHODS AND MATERIALS FOR DETECTING FRAGILE X MUTATIONS

(75) Inventors: William E. Highsmith, JR., Rochester, MN (US); Nicole L. Hoppman-Chaney, Rochester, MN (US); Malinda L. Butz, Chatfield, MN (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/726,092

(22) Filed: Mar. 17, 2010

Related U.S. Application Data
(60) Provisional application No. 61/162,755, filed on Mar. 24, 2009.

(51) Int. Cl. C12Q 1/68

(52) U.S. Cl. 435/6

(57) ABSTRACT

This document relates to methods and materials involved in detecting Fragile X mutations and assessing the methylation state of Fragile X alleles. For example, methods and materials for detecting Fragile X alleles using polymerase chain reaction and a hybridization probe (e.g., a non-radioactively labeled hybridization probe) are provided.

### CROSS-REFERENCE TO RELATED APPLICATION

This application claims priority to U.S. Provisional Application No. 61/162,755, filed on Mar. 24, 2009, the contents of which are incorporated by reference herein in their entirety.

### BACKGROUND

1. Technical Field

This document relates to methods and materials involved in detecting Fragile X mutations and assessing the methylation state of Fragile X alleles. For example, this document provides methods and materials for detecting Fragile X alleles using polymerase chain reaction and a hybridization probe (e.g., a non-radioactively labeled hybridization probe).

2. Background Information

Fragile X (FX) syndrome is an X-linked mental retardation syndrome that is associated with moderate to severe mental retardation, behavioral problems such as hyperactivity attention deficit and autism spectrum disorders, and a characteristic appearance. The causative mutation for FX syndrome is the expansion of the number of trinucleotide (CGG) repeats in the 5′ untranslated region of the FX mental retardation gene (FMR1) from a normal range of 5-44 CGG repeats to over 200 CGG repeats. Males with the mutation almost always exhibit the clinical phenotype, albeit of variable severity. Some females are clinically affected, exhibiting mental retardation and behavioral problems. Others, however, may exhibit milder learning disabilities or may be completely asymptomatic. The mechanism of disease is believed to be that the expanded CGG repeat and the flanking promoter region become heavily methylated which inactivates transcription of the FMR1 gene.

### SUMMARY

This document relates to methods and materials for detecting Fragile X mutations. This document is based, in part, on the discovery of nucleic acid detection assays that can be used to genotype individual Fragile X alleles having expanded trinucleotide repeat regions without the use of the time- and labor-intensive genomic Southern transfer techniques. As described herein, this document provides, for example, methods and materials by which a clinician or other professional can perform a polymerase chain reaction followed by a hybridization reaction to detect Fragile X mutations. In some cases, the hybridization can be performed without the use of radioactivity. In some cases, detection of normal and expanded trinucleotide repeat regions can be performed by capillary electrophoresis of fluorescently-labeled PCR products only, without the need for hybridization. This document also provides methods and materials by which a clinician or other professional can assess and classify the methylation state of Fragile X full mutant alleles, Fragile X pre-mutation alleles, Fragile X intermediate mutant alleles, or non-mutant Fragile X alleles.

In general, one aspect of this document features a method for assessing Fragile X alleles in a mammal. The method comprises, or consists essentially of, amplifying nucleic acid obtained from a mammal to obtain amplified nucleic acid. The amplified nucleic acid can comprise a sequence from a regulatory region for a Fragile X polypeptide-encoding sequence. A Fragile X polypeptide-encoding sequence can comprise a CGG location flanked by a 3′ non-CGG sequence and a 5′ non-CGG sequence. The method further comprises, or consists essentially of, detecting the size of an amplified nucleic acid using a Southern blot and an oligonucleotide probe. An oligonucleotide probe can hybridize to a repeated CGG sequence of an amplified nucleic acid. The presence of a size diagnostic of greater than 200 CGG repeats at a CGG location in an amplified product can indicate that a mammal comprises a Fragile X full mutant allele. The presence of a size diagnostic of 59 to 199 CGG repeats at a CGG location in an amplified product can indicate that a mammal comprises a Fragile X pre-mutation allele. The presence of a size diagnostic of 45 to 58 CGG repeats at a CGG location in an amplified product indicates that a mammal comprises a Fragile X intermediate mutant allele. The presence of a size diagnostic of less than 45 CGG repeats at a CGG location in an amplified product indicates that a mammal comprises a non-mutant Fragile X allele. The mammal can be a human. The nucleic acid can be genomic DNA. The method can also comprise classifying a mammal as having a Fragile X full mutant allele, a Fragile X pre-mutation allele, a Fragile X intermediate mutant allele, or a non-mutant Fragile X allele. The method can also comprise assessing a methylation state of a regulatory region for a Fragile X polypeptide-encoding sequence by digesting a nucleic acid with a methylation-sensitive restriction enzyme prior to the amplifying step. In some cases, amplification occurs when the nucleic acid is methylated at a recognition site for the methylation-sensitive restriction enzyme, and amplification does not occur when the nucleic acid is not methylated at a recognition site for the methylation-sensitive restriction enzyme.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains photographs of (A) an ethidium bromide-stained agarose gel and (B) a nylon membrane following blotting. PCR amplification products were separated according to size by agarose gel electrophoresis. Blotting was performed using a non-radioactively labeled hybridization probe. Lanes 2, 12, 24: full mutation males. Lanes 4-10, 13-16, 18-22: full mutation females. Lane 3: male mosaic for full mutation and pre-mutation alleles. Lane 17: male mosaic for full mutation and normal alleles. Lane 1: female mosaic for full mutation and pre-mutation. Lanes 23, 26: female pre-mutation carriers. Lanes 11, 25, 27, 28: normal females.

FIG. 2 contains photographs of (A) a genomic DNA Southern blot and (B) a post-PCR Southern blot. Samples were male full Fragile X mutation DNA diluted to final concentrations of 90%, 70%, 50%, 40%, 30%, 20%, and 10% in male normal DNA.

FIG. 3 contains photographs of post-PCR Southern blots. Each membrane was probed with an oligonucleotide probe labeled using the Amersham Gene Images AlkPhos Direct Labeling kit.

FIG. 4 contains a summary of the results of post-PCR Southern blotting as compared to clinical, genomic DNA Southern blotting results. 200 samples were assayed, 197 of which gave results.

FIG. 5 contains capillary electropherograms using both the ABI 3130 and the ABI 3730 instruments for a sample obtained from a male who has a full mutation and a small amount of a normal, 28 repeat allele (i.e. the male is a mosaic for a normal allele and a full mutation). The full mutation is detected using the ABI 3730 instrument, but not using the ABI 3130 instrument.

FIG. 6 contains a summary of the results of ABI 3730x1 capillary electrophoresis analysis as compared to clinical, genomic DNA Southern blotting with radioactive PCR results. 190 samples were assayed, 187 of which gave results.

FIG. 7 is a nucleic acid sequence (SEQ ID NO:1) sequence of the region immediately upstream of the FMR1 gene. Intron sequence is in lower case text. Exon sequence is in upper case text. Five Hha I recognition sequences are double-underlined. One BsaHI recognition sequence is boxed. Forward primer and reverse primer binding sequences are underlined.

FIG. 8 contains photographs of ethidium bromide stained agarose gels. (A) Undigested post-PCR product. (B) Hha I-digested post-PCR product. Lanes 1, 11, 13, 23: full mutation females. Lanes 2, 4, 12, 18, 20, 21: normal females. Lanes 3, 10, 15, 22: pre-mutation females. Lanes 5, 14, 17, 19: full mutation males. Lanes 6, 8, 24: normal males. Lanes 7, 16: pre-mutation males.

FIG. 9 contains photographs of blots of (A) undigested post-PCR products and (B) Hha I digested post-PCR products. Blotting was performed using a non-radioactively labeled hybridization probe. Lanes are as identified in FIG. 8.

FIG. 10 contains photographs of post-PCR Southern blots. Genomic DNA samples were digested with Hha I prior to amplification by PCR. Each membrane was probed with an oligonucleotide probe labeled using the Amersham Gene Images AlkPhos Direct Labeling kit.

FIG. 11 contains a summary of the results of pre-PCR Hha I digestion and post-PCR Southern blotting as compared to clinical, genomic DNA Southern blotting results. 158 samples were assayed, 154 of which gave results. Genomic DNA samples were digested with Hha I prior to amplification by PCR.

FIG. 12 contains a photograph of an ethidium bromide stained agarose gel. (A) Undigested post-PCR product. (B) Hha I-digested post-PCR product. (C) Sfo I-digested post-PCR product. (D) BsaHI-digested post-PCR product. (E) BstUI-digested post-PCR product. Lanes 1, 7, 8: pre-mutation males. Lane 2: normal female. Lane 3: normal/full mutation mosaic male. Lane 4: pre-mutation female. Lane 5: full mutation female. Lane 6: full mutation male. Lane M: size marker (pGEM).

FIG. 13 contains a photograph of blots of (A) Undigested post-PCR product, (B) Hha I-digested post-PCR product, (C) Sfo I-digested post-PCR product, (D) BsaHI-digested post-PCR product, and (E) BstUI-digested post-PCR product. Blotting was performed using a non-radioactively labeled hybridization probe. Lanes are as identified in FIG. 12.

FIG. 14 contains photographs of capillary electrophoresis of (A) undigested and (B) Hha I-digested Fragile X PCR products of a premutation male on the ABI 3730x1.

FIG. 15 contains a summary of the results of pre-PCR BsaHI digestion and post-PCR Southern blotting as compared to clinical, genomic DNA Southern blotting results. 85 samples were assayed and all gave results. Genomic DNA samples were digested with BsaHI prior to amplification by PCR with subsequent post-PCR blotting and hybridization with a non-radioactively labeled probe.

### DETAILED DESCRIPTION

This description provides methods and materials related to determining whether or not a mammal contains a full mutation, a pre-mutation, or a normal allele at the Fragile X locus. The document also provides a method for detecting the Fragile X genotype of a mammal.

The methods and materials provided herein can be used to detect normal, pre-mutation, and full mutation Fragile X alleles in a mammal. The mammal can be any type of mammal, including, without limitation, a mouse, rat, dog, cat, horse, goat, cow, pig, monkey, or human. For example, the methods and materials provided here can be used to assess the Fragile X genotype of a human patient. In some cases, Fragile X mutations can be detected in a mammal using genomic DNA. Genomic DNA can be extracted from any biological sample containing nucleated cells, such as a peripheral blood sample or a tissue sample (e.g., mucosal scrapings of the lining of the mouth) or from other bodily fluids. In some cases, genomic DNA can be extracted from microdissected tissue samples or histology slides. In some cases, genomic DNA can be extracted from embryonic tissue, polar body of a preimplantation embryo, a two-cell stage embryo, a four-cell stage embryo, an eight-cell stage embryo, a 16-celled embryo, a 32-celled embryo, a 64-celled embryo, a 132-celled embryo, a 256-celled embryo, a 512-celled embryo, a 1024-celled embryo, or cells from a fetus, neonate, or child. Standard methods can be used to extract genomic DNA from a blood or tissue sample, including, for example, phenol extraction. In some cases, genomic DNA can be extracted with kits such as the QIAamp® Tissue Kit (Qiagen, Valencia, Calif.) and the Wizard® Genomic DNA purification kit (Promega, Madison, Wis.). In some cases, biological samples can be obtained by a clinician or other professional and sent to a diagnostic or research laboratory to extract genomic DNA and to detect Fragile X alleles as provided herein.

Once obtained, a genomic DNA sample can be amplified to obtain detectable levels of amplified nucleic acid. Any appropriate method can be used to amplify the genomic DNA. For example, polymerase chain reaction (PCR) can be used to amplify specific sequences from DNA, including sequences from total genomic DNA. PCR refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. See e.g., PCR Primer: A Laboratory Manual, Dieffenbach and Dveksler, (Eds.); McPherson et al., PCR Basics: From Background to Bench (Springer Verlag, 2000); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. Amplified nucleic acid can have Fragile X CGG trinucleotide repeats and can be assessed or detected as described herein for amplified products. In some cases, amplification can be performed using a commercially available amplification kit. For example, amplification can be performed using a Fragile X PCR kit (Celera/Abbott) according to protocols supplied by the manufacturer.

Any appropriate oligonucleotide primers can be used to amplify FX alleles according to the methods provided herein. In some cases, Fragile X allele specific oligonucleotide primers can be used to amplify the genomic DNA. For example, Fragile X allele specific primers can be designed according to methods provided in U.S. Provisional Patent Application Ser. No. 61/113,894. In some cases, allele-specific primers can be used to amplify at least a portion of the Fragile X region. Primers can be 14 to 40 nucleotides in length, or longer (e.g., up to 100, 200, or more nucleotides in length). In some cases, primers can be between about 20 and about 30 nucleotides in length. General PCR techniques are described, for example, in PCR Primer: A Laboratory Manual, ed. by Dieffenbach and Dveksler, Cold Spring Harbor Laboratory Press, 1995. In some cases, a forward primer can have the sequence 5′-GAC GGA GGC GCC GCT GCC AGG-3′ (SEQ ID NO:2), and a reverse primer can have the sequence 5′-GTG GGC TGC GGG CGC TCG AGG-3′ (SEQ ID NO:3). See, for example, Wilson et al., J. Mol. Diagn. 10(1):2-12 (2008). In other cases, a forward primer can have the sequence 5′-TCA GGC GCT CAG CTC CGT TTC-3′ (SEQ ID NO:5), and a reverse primer can have the sequence 5′-CCT CCA TCT TCT CTT CAG CCC TG-3′ (SEQ ID NO:6).

In some cases, a genomic DNA sample can be digested with a methylation-sensitive restriction endonuclease prior to amplification. Any restriction endonuclease that is unable to cleave if the enzyme recognition sequence contains a methylated base can be used. For example, genomic DNA can be digested with restriction endonucleases Hha I or BsaHI, and the digested nucleic acid can be used for amplification as described herein.

Once obtained, amplified nucleic acid containing Fragile X CGG trinucleotide repeats can be detected according to the methods provided herein. In some cases, amplified nucleic acid can be identified directly with a nucleic acid specific stain. Any appropriate nucleic acid stain, such as ethidium bromide, SYBRGreenI®, or SYBRGold® nucleic acid binding dyes, can be used. For example, amplified PCR product can be detected by performing gel electrophoresis in an agarose gel dyed with ethidium bromide. Other suitable nucleic acid binding dyes that can be used for the methods and materials provided herein can include acridine orange, DAPI, bisbenzimide, BlueView® nucleic acid stain (Sigma-Aldrich), and dimeric cyanine nucleic acid dyes (Molecular Probes).

In some cases, amplified nucleic acid containing Fragile X CGG trinucleotide repeats can be detected using a polynucleotide hybridization probe. Hybridization methods, such as Southern analysis or Northern analysis can be used (see Ausubel et al., Current Protocols in Molecular Biology, eds., John Wiley & Sons (2003)). For example, amplified nucleic acids can be transferred to a membrane according to Southern blotting techniques, and a probe can be hybridized to amplified nucleic acids transferred to the membrane. The hybridization probe can be RNA or DNA. In some cases, the hybridization probe can be at least substantially complementary to a gene. In some cases, a hybridization probe can specifically hybridize to Fragile X sequences. For example, a hybridization probe can have the nucleotide sequence as set forth in SEQ ID NO:4. In order to detect hybridization of the probe to transferred nucleic acids, the hybridization probe can be labeled. Any appropriate label can be used. For example, a hybridization probe can be labeled with a fluorescent label (e.g., fluorescein). In some cases, a hybridization probe can be labeled with a Gene Images AlkPhos Direct Labeling kit (Amersham). In some cases, a hybridization probe can be labeled with a molecule that produces a color precipitate (e.g., alkaline phosphatase, digoxigenin) following a colorization reaction. Other probe labels suitable for the methods described herein can include radioisotope tags, electrochemiluminescent (ECL) labels, or biotin.

In some cases, Fragile X alleles can be detected without a manual blotting step. For example, amplification of genomic DNA can include fluorescently-labeled oligonucleotide primers. In some cases, the resulting labeled PCR product can be purified using magnetic beads (e.g., AMPure beads (Beckman Genomics Inc.)) or other purification methods. The resulting labeled PCR product can be analyzed by capillary electrophoresis with a laser-induced fluorescence detector (CE-LIF) (Applied Biosystems). In other instances, once obtained, amplified nucleic acid can be hybridized with a fluorescently labeled probe, the probe can be labeled with a different fluorescent dye than the PCR primers, and the resulting double stranded product can be analyzed by CE-LIF. In some cases, the hybridization probe can be a peptide nucleic acid (PNA).

In some cases, amplification of genomic DNA can include fluorescently-labeled nucleotides. In such cases, the resulting labeled PCR product can be analyzed using capillary electrophoresis with a laser-induced fluorescence detector. Any appropriate capillary electrophoresis instrument can be used. In some cases, a capillary electrophoresis instrument can have increased fluorescence sensitivity. In some cases, a capillary electrophoresis instrument can have an increased dynamic signal range. For example, the resulting labeled PCR product can be analyzed using an ABI 3730x1 capillary electrophoresis instrument (Applied Biosystems). In some cases, the ABI 3730x1 capillary electrophoresis instrument can have a larger dynamic range for signal than an ABI 3130x1 capillary electrophoresis instrument (Applied Biosystems) (e.g., 0-30,000 RFU versus 0-16,000 RFU, respectively). In some instances, the ABI 3730x1 capillary electrophoresis instrument also can have increased fluorescence sensitivity relative to an ABI 3130x1 capillary electrophoresis instrument.

In some cases, detecting an amplified product of a particular size can indicate the presence and/or identity of normal, intermediate, pre-mutation, or full Fragile X alleles. For example, the presence of amplified product indicative of greater than 200 CGG repeats at a CGG location can indicate that a mammal has at least one Fragile X full mutant allele; the presence of amplified product indicative of between 59 to 199 CGG repeats at a CGG location can indicate that a mammal has at least one Fragile X pre-mutation allele; the presence of amplified product indicative of between 45 to 58 CGG repeats at a CGG location can indicate that a mammal has at least one Fragile X intermediate mutant allele, and the presence of amplified product indicative of less than 45 CGG repeats at a CGG location can indicate that a mammal has at least one non-mutant Fragile X allele.

In some cases, detection of normal, intermediate, pre-mutation, or full Fragile X alleles can enable clinicians or other professionals to classify a mammal as possessing a Fragile X full mutation allele, a Fragile X pre-mutation allele, a Fragile X intermediate mutation allele, and/or a Fragile X non-mutant allele based on the presence or absence of a given number of CGG trinucleotide repeats. In some cases, results of the mutation detection methods and materials provided herein can be communicated by research technicians or other professionals who perform the amplification to clinicians or other professionals who will classify the mammal as having a particular Fragile X genotype based on the presence or absence of a given number of CGG trinucleotide repeats. For example, a research technician or other professional who perform the amplification can refer a subject to a clinician or other medical professional on the basis of these classifications. In some cases, clinicians or other professionals can evaluate and recommend appropriate treatment options, educational programs, and/or other therapies with the goal of optimizing patient care. In some cases, clinicians or other professionals can initiate or modify a course of treatment after receiving information regarding a determination that a patient has a particular Fragile X genotype, for example.

After classifying the mammal as having a particular Fragile X genotype based on the presence or absence of a given number of CGG trinucleotide repeats, a health-care professional can take one or more actions that can affect the mammal's care. For example, a health-care professional can record information regarding the mammal's Fragile X genotype in the mammal's medical record. In some cases, a health-care professional can record that the mammal has Fragile X or is a carrier of Fragile X, or otherwise transform the mammal's medical record, to reflect the mammal's medical condition. For example, a clinician or other professional can make a permanent notation or flag a medical record for communicating the mammal's Fragile X genotype to other professionals reviewing the record. In addition, any type of communication can be used to communicate the mammal's Fragile X genotype. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional. In some cases, a health-care professional can review and evaluate a mammal's medical record, and can assess multiple treatment strategies for clinical intervention of a mammal's condition. Genetic counseling with regards to the probability of having children with the Fragile X syndrome can also be given.

The invention will be further described in the following examples, which do not limit the scope of the invention described in the claims.

### EXAMPLES

### Example 1

### Detection of Full Fragile X Mutations by Post-PCR Southern Blot

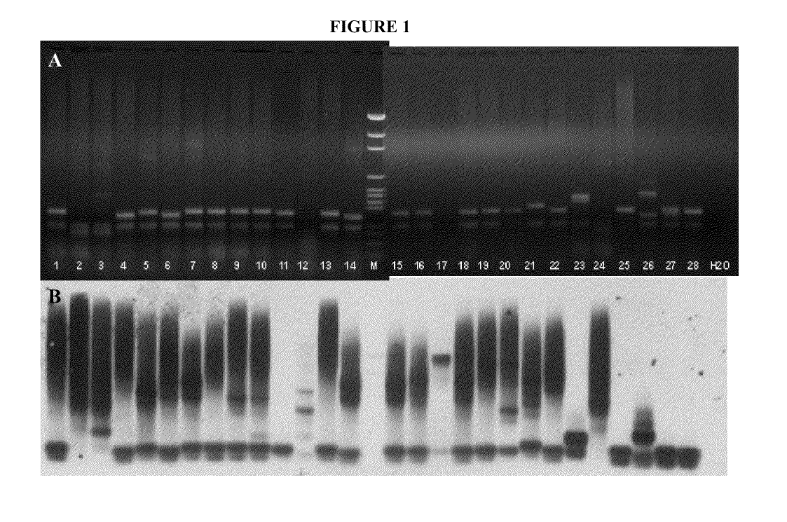

In order to test the notion that full mutations could be detected using a non-radioactive post-PCR Southern blot, 29 human genomic DNA samples were amplified using reagents and protocols provided with the Fragile X PCR kit (Celera/Abbott). PCR products were electrophoresed on an agarose gel according to the kit instructions. The gel was stained with ethidium bromide and photographed using UV transillumination (FIG. 1A). The PCR products were transferred to a nylon membrane using standard Southern blotting methods. The membrane was probed with a non-radioactive oligonucleotide probe with the sequence: 5′-CCG CCG CCG CCG CCG CCG CCG-3′ (SEQ ID NO:4). The probe was labeled using a Gene Images AlkPhos Direct Labeling kit (Amersham). The membrane was probed overnight, and then subjected to a final washing stringency of 1× Secondary Wash Buffer (Amersham) for 10 minutes at room temperature. After high stringency washing, the membrane was treated with the detection reagents from the labeling kit, and exposed to X-ray film for one hour. A photograph of the developed film was taken using the Alpha Imager (FIG. 1B).

Included in this experiment were males with full mutations (FIG. 1B, lanes 2, 12, 24), males mosaic for full mutations and pre-mutations (lane 3) or normal alleles (lane 17); females with full mutations (lanes 4-10, 13-16, 18-22), a female mosaic for a pre- and a full mutation (lane 1); female pre-mutation carriers (lanes 23, 26); and normal females (lanes 25, 27, 28). A light signal was observed for the full mutation male in lane 12, a CVS sample that had a very high DNA concentration. It is possible that the proper amount of DNA was not added to the PCR reaction due to viscosity. A smear was apparent for the normal female in lane 25 by ethidium bromide-stained gel electrophoresis which was not present post-PCR blot. In the absence of a post-PCR Southern blot (SB), such a result would have been scored as a false positive. Taken together, the results of the post-PCR SB with the labeled probe described herein indicate 100% concordance with the clinical, genomic DNA Southern blot results for these 29 samples. A second set of 25 samples also showed 100% sensitivity and 100% specificity compared to the clinical results.

### Example 2

### Sensitivity for Mosaicism

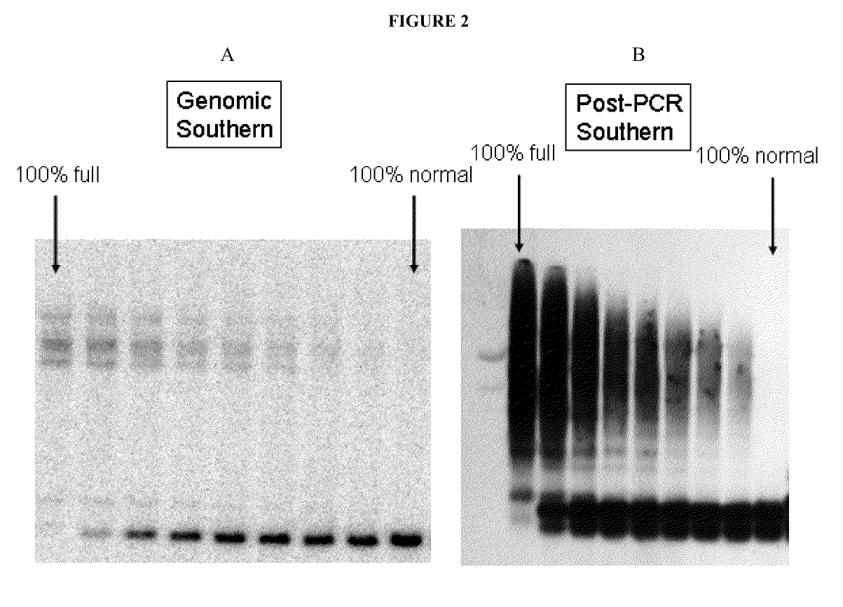

The results of the post-PCR Southern blot revealed that three of the mosaic cases showed quite robust expanded alleles. To further investigate the sensitivity of the post-PCR SB method for the detection of mosaic cases, a set of samples were prepared that had DNA from a full mutation male combined with DNA from a normal male. Male full Fragile X mutation DNA diluted to final concentrations of 90%, 70%, 50%, 40%, 30%, 20%, and 10% in male normal DNA were tested. Both genomic Southern blotting and the post-PCR SB assays were carried out (FIG. 2). The full mutation was easily detectable at a 1:9 dilution in the post-PCR SB, whereas the detection limit for the genomic SB was approximately 20-30%. Thus, the post-PCR SB method had full sensitivity for full mutation mosaicism equal to, or better than, genomic SB.

### Example 3

### Validation of Post-PCR Blotting Method

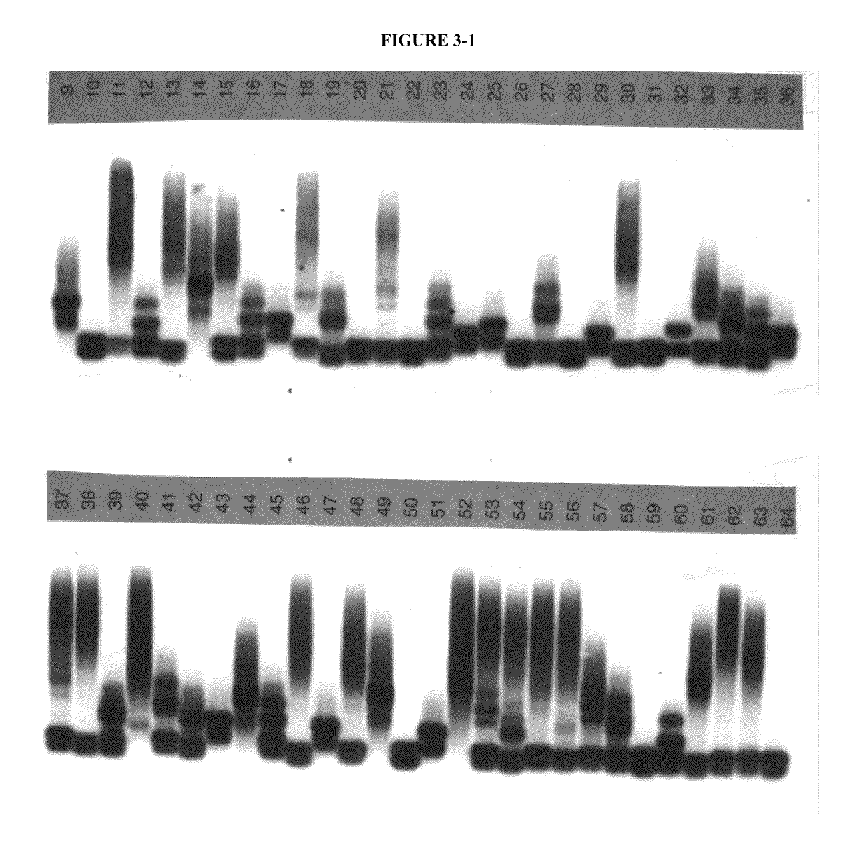

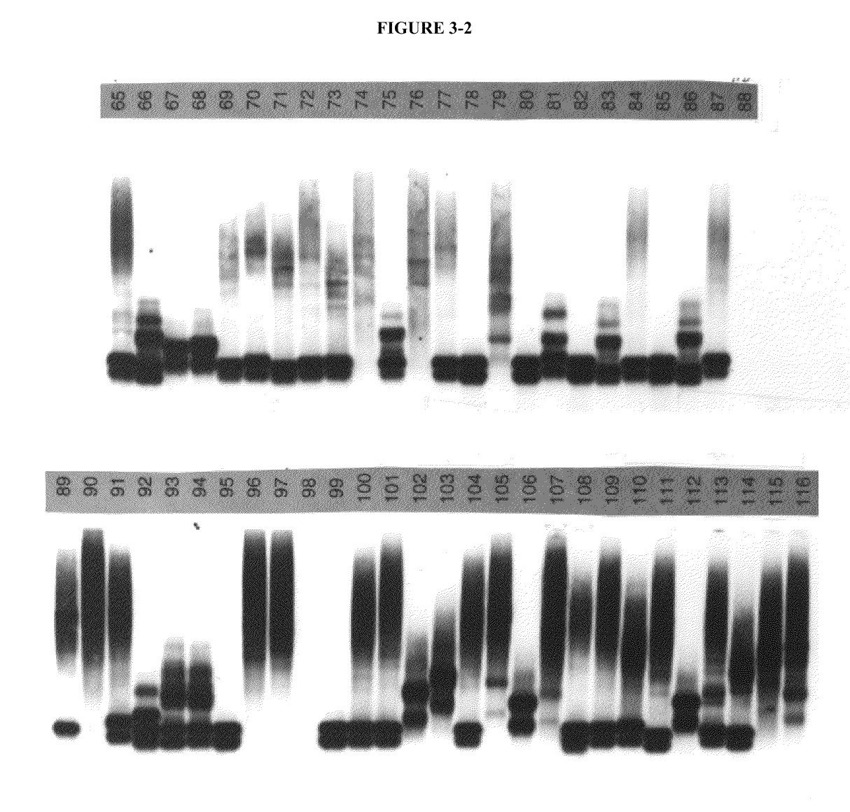

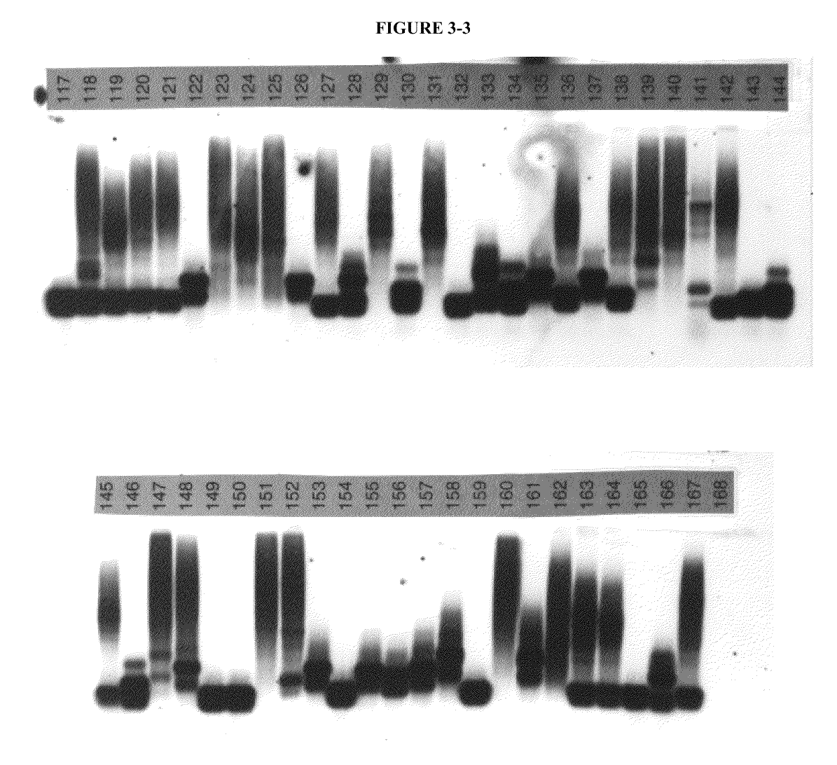

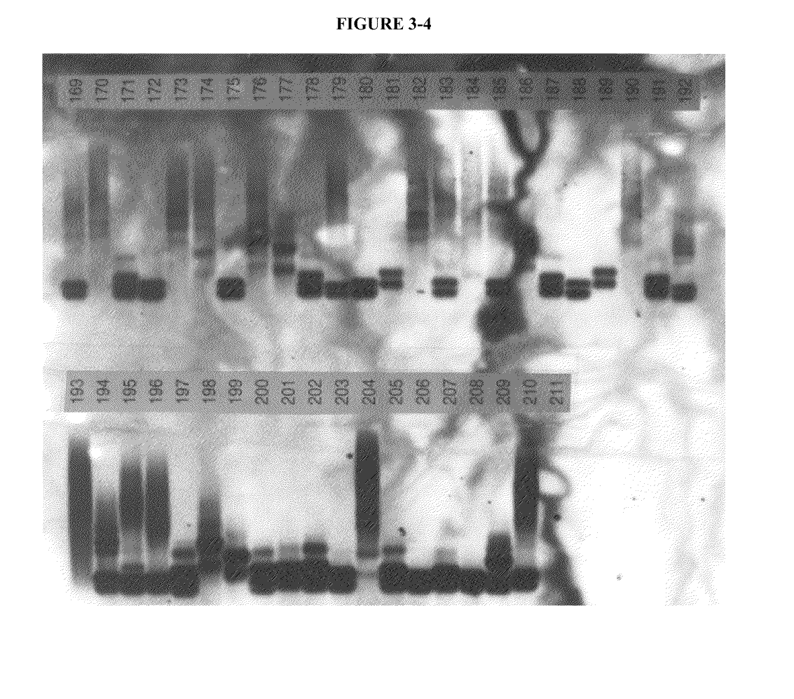

In order to validate the Post-PCR Southern blot method, 200 samples that were previously tested in our laboratory by the genomic Southern blot and radioactive PCR methods were amplified and blotted in a “blinded” fashion. PCR amplification was performed using the Fragile X PCR kit (Celera/Abbott) according to the protocols supplied by the manufacturer. The PCR products were electrophoresed on an agarose gel. After taking a photo of the ethidium bromide stained gel using UV transillumination, the PCR products were transferred to a nylon membrane using standard SB methods. The membrane was probed with a non-radioactive oligonucleotide probe having the following nucleotide sequence: 5′ CCG CCG CCG CCG CCG CCG CCG 3′ (SEQ ID NO:4). The probe was labeled using the Amersham Gene Images AlkPhos Direct Labeling kit and hybridized for four hours. The membrane was subjected to a final high stringency wash with 1× Secondary Wash Buffer (Amersham) for 10 minutes at room temperature. After washing, the membrane was treated with the CDP-Star chemilluminescent detection reagent (Amersham), and exposed to X-ray film for one hour. A photograph of the developed film was taken using the Alpha Imager (FIG. 3).

Included in the validation experiment was a mixture of 70 full mutation carrier females and affected males. 56 pre-mutation and 12 intermediate allele carriers were also included. To check for potential false positives, 31 samples previously identified as having normal alleles at the Fragile X locus were included. Most importantly, 31 samples were mosaic for size (e.g., having combinations of full, pre-mutation, intermediate, and normal alleles) and/or methylation.

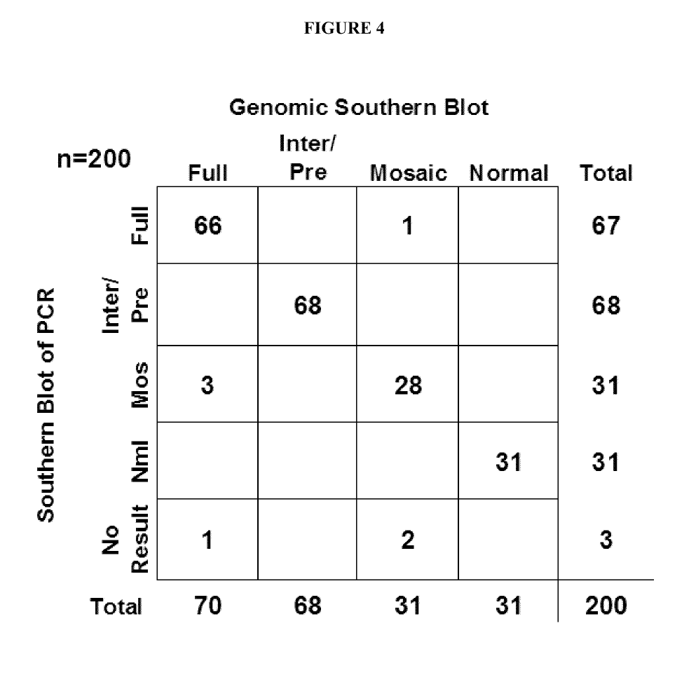

For the 200 samples analyzed for the validation of Southern blot of the Celera/Abbott Fragile X PCR, 98.0% concordance with the clinical, genomic DNA Southern blot results were observed for the 197 samples with results (FIG. 4). Three of the four discrepancies were due to increased sensitivity for mosaicism using the Celera/Abbott PCR with SB. The remaining discrepant sample was methylation mosaic, which this assay cannot detect; however, the full mutation was detected. Overall, the analysis showed 100% sensitivity and 100% specificity for discriminating normal from abnormal samples, when compared to previous testing results from our laboratory.

### Example 4

### Detection of Full Fragile X Mutations by Capillary Electrophoresis Alone

It was discovered that full Fragile X mutations could be detected by capillary electrophoresis alone (without post-PCR blotting). It was observed that full mutations were detectable with strong, robust signals when fluorescently-labeled PCR products obtained using the Celera/Abbott Fragile X PCR analyte specific reagents were subjected to analysis on an ABI 3730x1 capillary electrophoresis instrument (Applied Biosystems), but not when the more commonly used ABI 3130x1 (Applied Biosystems) was used. In order to test the accuracy, sensitivity, specificity, and reliability of this process, 190 human genomic DNA samples were amplified using reagents and protocols provided with the Fragile X PCR analyte specific reagents (ASRs) (Celera/Abbott). PCR products were electrophoresed on the ABI 3730x1 DNA Analyzer using the double injection method suggested by the manufacturer. In this method, the fluorescently-labeled PCR product is injected into the capillary electrophoresis instrument twice: a short injection and a long injection. First, an injection was performed using an injection voltage of 8,000 volts for 1 second and completing the electrophoresis by running the instrument at 8,500 volts for 3300 seconds. This procedure (short injection) allowed analysis of normal size alleles.

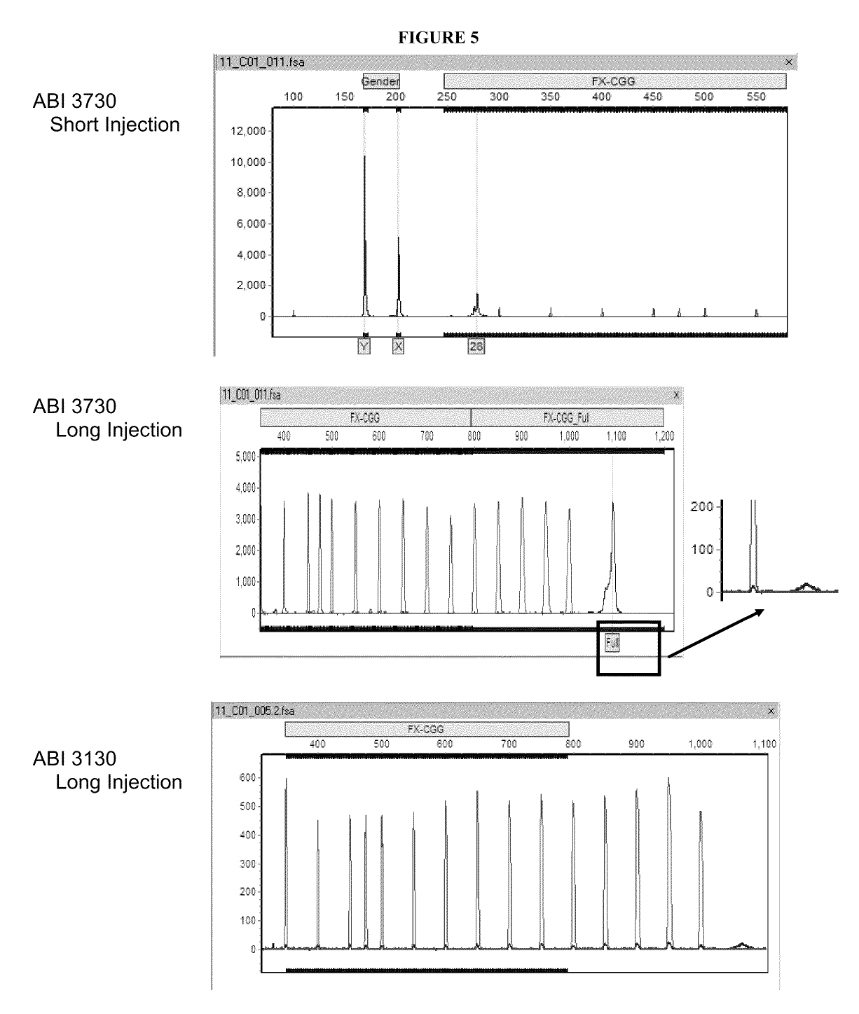

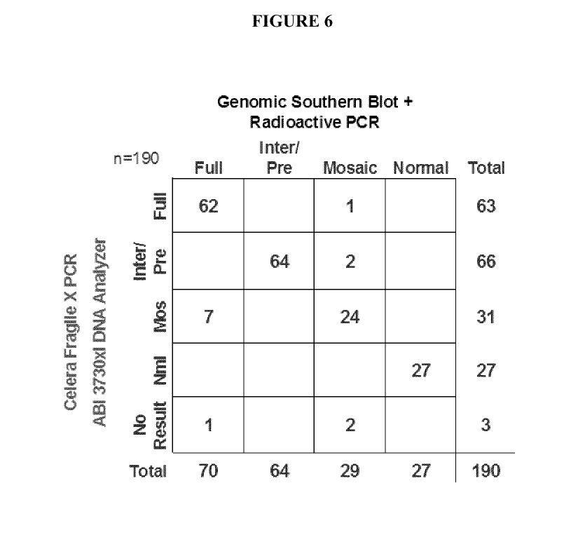

After the first analysis was completed, the sample was reinjected at a voltage of 8,000 volts for 22 seconds and electrophoresed at 8,500 volts for 3,800 seconds. This procedure (long injection) loads more PCR product on the capillary electrophoresis column and allows for detection of permutation and full mutation alleles. Results were obtained on 187 of 190 samples. Included in this experiment were 70 males and females with full mutations, 64 male and female premutation carriers, 29 individuals mosaic for full mutations and pre-mutations or normal alleles, and 27 normal females. Results were obtained for 69 of 70 full mutation samples (FIG. 5). All 69 (100%) full mutation alleles were detected; with 7 of the samples classified as “mosaic” due to additional (premutation) alleles detected that were not detected by Southern blot. This was due to the increased sensitivity of the ABI 3730x1 method compared to the reference Southern blot method. Three methylation mosaic samples, as previously determined by Southern blot, were misclassified as either full mutation or premutation samples (FIG. 5). However, this assay is not designed to detect methylation mosaicism. When analyzing data obtained from the ABI 3730x1, 100% sensitivity (no false negatives) and 100% specificity (no false positives) were observed as compared to the reference Southern blot with radioactive PCR method. These results indicate 100% concordance with genomic DNA Southern blot results for detecting expanded FX mutation alleles in these 187 samples (FIG. 6). This ABI 3730x1 method was further improved to reduce repeat analyses. The improvement involves doing a purification of the FX PCR using AMPure reagent before capillary electrophoresis on the ABI 3730x1. This modification decreased the sample failure rate from approximately 4-14% down to less than 1%.

### Example 5

### Detection of Methylation State with Hha I

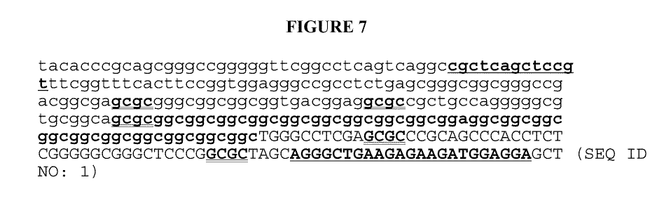

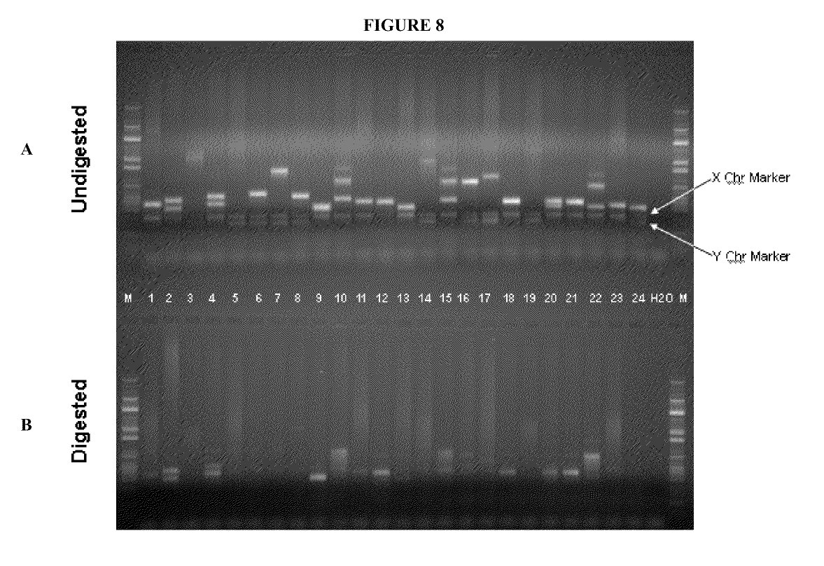

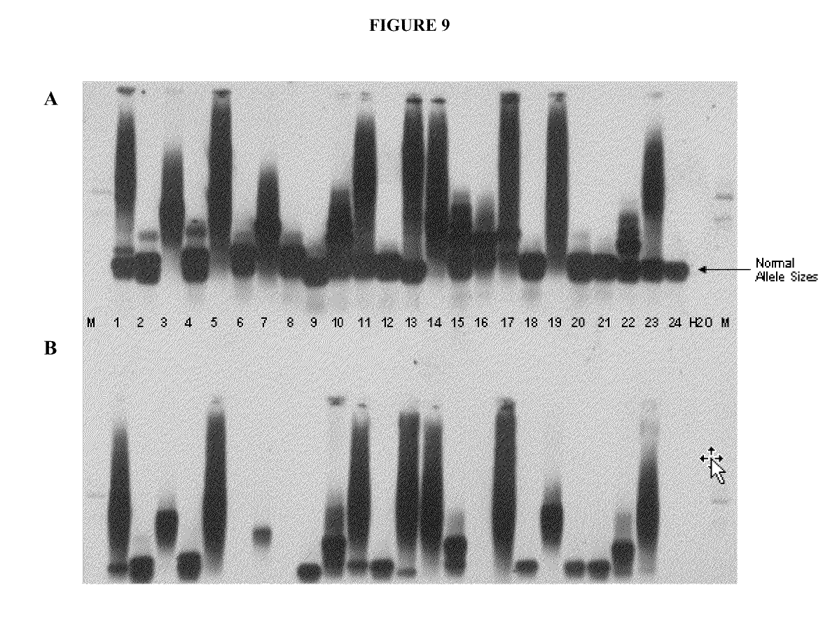

The pathogenic event causing failure of FMR1 transcription is not the CGG expansion per se, but rather the subsequent methylation of the surrounding promoter region. Although PCR based methylation assays have been reported, the genomic SB is typically used to assess the methylation state of the FMR1 gene. It was hypothesized that the post-PCR SB method could be modified to detect the methylation state of Fragile X alleles. The region surrounding the CGG repeat (FIG. 7, in bold) has several GCGC recognition sites for the methylation-sensitive restriction endonuclease Hha I (FIG. 7). Five Hha I recognition sequences are located between the primers used in the methods described herein (FIG. 7). Hha I cleaves double stranded DNA at the GCGC recognition sequence only if the cytosine residues are unmethylated. Thus, it was hypothesized that digestion of genomic DNA with Hha I prior to PCR according to the Fragile X PCR kit would allow amplification of only methylated sequences. To test this hypothesis, genomic DNA was digested with Hha I. The restriction endonuclease-treated DNA was amplified using the Fragile X PCR kit (Celera/Abbott). PCR products were size separated on an agarose gel stained with ethidium bromide (FIG. 8). PCR products were transferred to a nylon membrane by Southern blotting (FIG. 9). Of the normal male samples (FIG. 9; lanes 6, 8, and 24, each contained an unmethylated, normal sized allele. Of the pre-mutation males (FIG. 9; lanes 7 and 16), which were expected to be completely unmethylated, one sample contained a small percentage of methylated material (lane 16). Normal female samples (FIG. 9; lanes 2, 4, 12, 18, 20, and 21) contained normal sized alleles, half of which were unmethylated and half of which were methylated. In the pre-mutation female samples (FIG. 9; lanes 3, 10, 15, and 22), 50% were, as expected, methylated alleles and 50% were unmethylated. In the full male mutation samples (FIG. 9; lanes 5, 14, 17, and 19), the alleles were completely methylated as expected. Finally, the female full mutation samples (FIGS. 9; 1, 11, 13, and 23) had partial to complete methylation of the full mutation with an unmethylated normal allele. Importantly, the results were 100% concordant with the clinical genomic SB results, and the expected results were obtained in each trial. In sum, the post-PCR protocol can be modified to provide methylation information that is equivalent to genomic DNA SB.

### Example 6

### Validation of FMR1 Methylation State Detection with Hha I

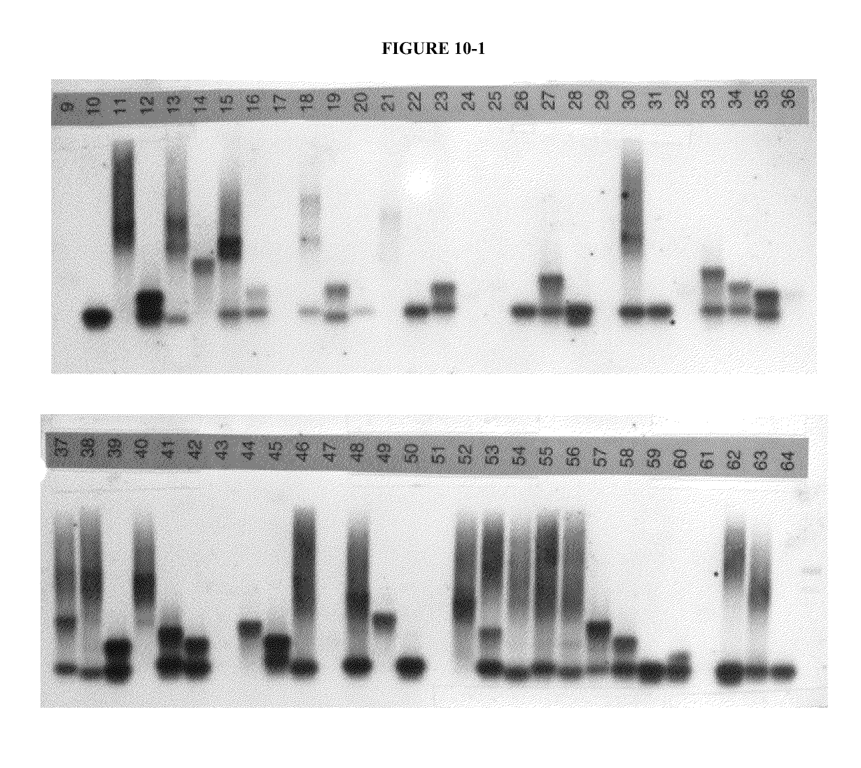

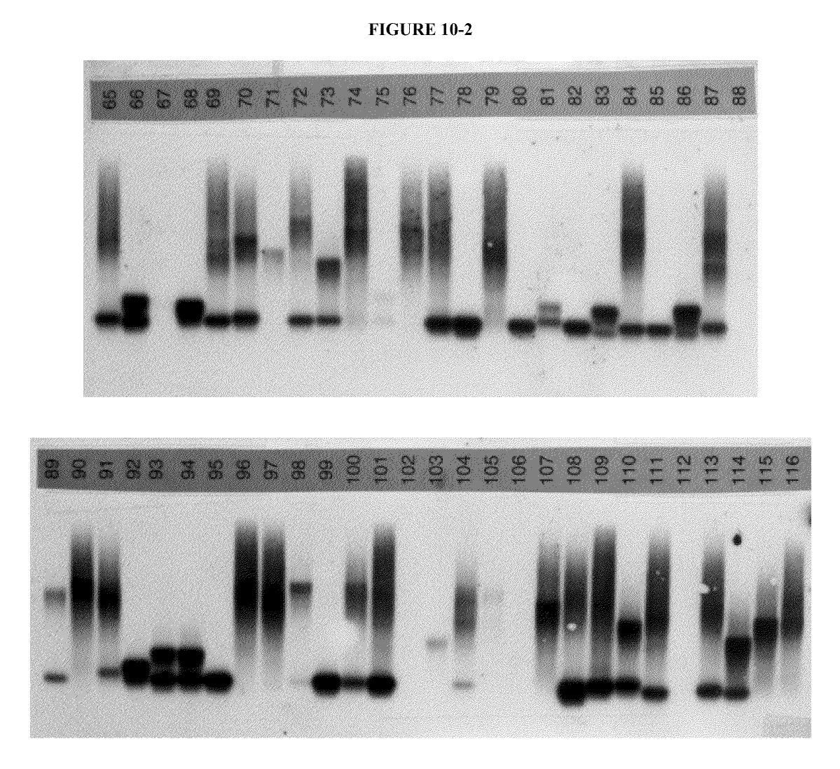

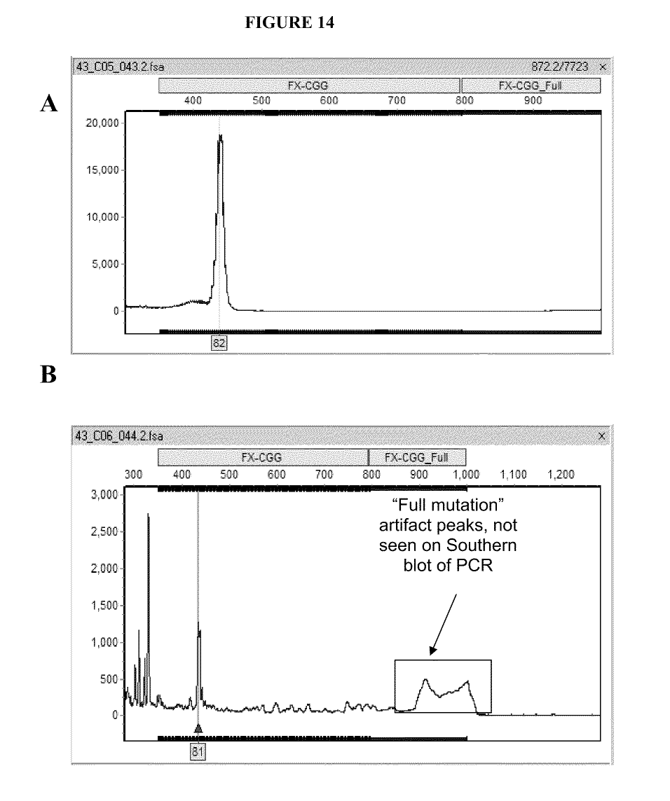

The purpose of the digestion of genomic DNA with Hha I methylation-sensitive restriction enzyme before the Celera/Abbot Fragile X PCR is to determine the methylation status of pre-mutation and full mutation FMR1 alleles, which is traditionally determined by genomic Southern blot. To validate this methylation state determination process, 157 samples from the sample set used for the undigested PCR Southern blot were digested with Hha I enzyme for 2 hours before amplification using the Celera/Abbott Fragile X PCR kit reagents. Following the PCR amplification, all procedures were performed as in the previous examples with the exception that the film was exposed to the blotted membrane for 2 hours instead of 1 hour. A photograph of each developed film was taken using the Alpha Imager (FIG. 10). All samples were also analyzed by capillary electrophoresis on the ABI 3730x1, to test the possibility of analyzing the data by this method, without the need for the post-PCR blot. However, some samples (˜25%) showed some type of artifact peak(s), often in the full mutation range, which did not appear on the blot (see FIG. 14). Further, it was not possible to distinguish mosaics from heterozygotes using capillary electrophoresis after amplification of Hha I digested genomic DNA.

Included in this experiment was a mixture of 59 full mutation carrier females and affected males. Fifty pre-mutation and 4 intermediate allele carriers were also included. To check for potential false positives, 23 normal samples, previously tested for Fragile X in the laboratory, were included. Most importantly, 22 samples mosaic for size (combinations of full, pre-mutation, intermediate, and normal alleles) and/or methylation were also assayed.

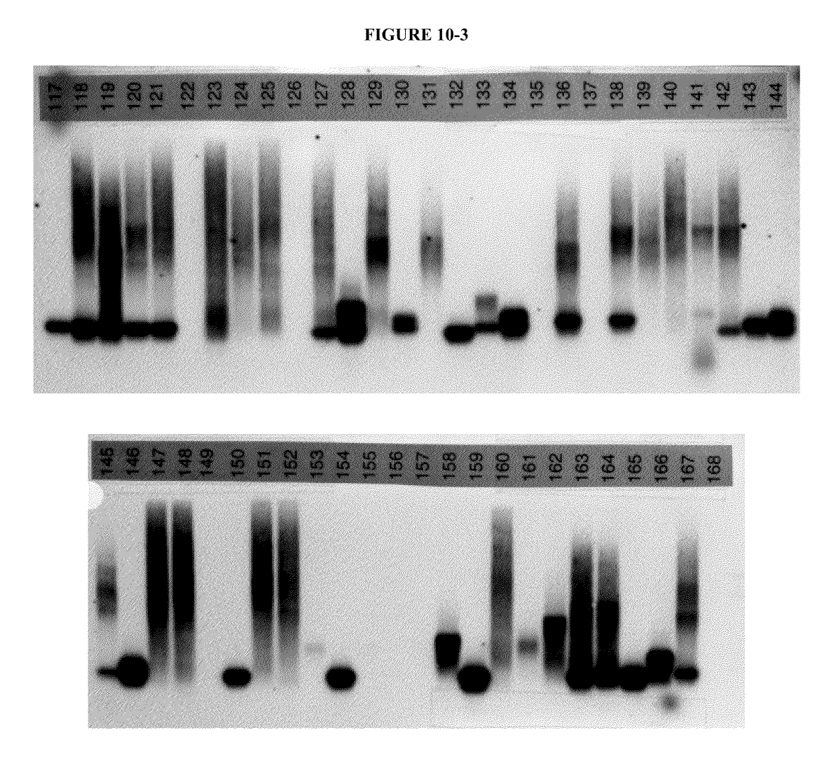

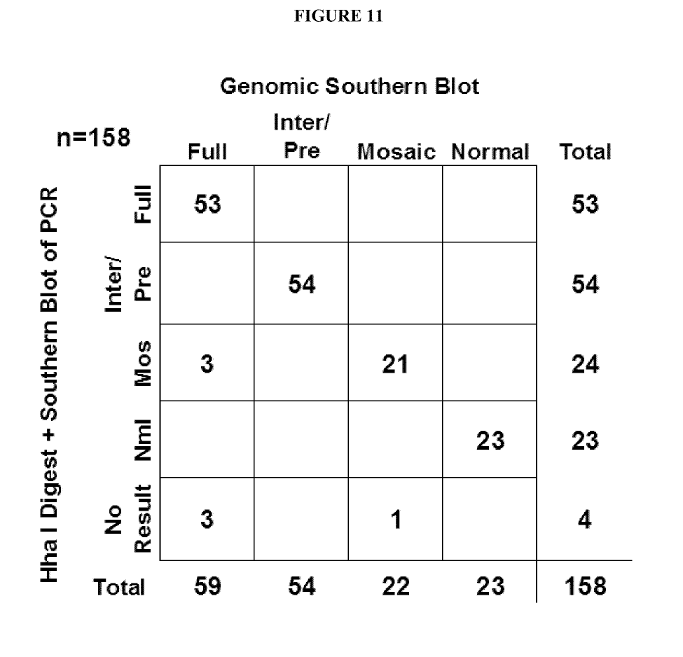

It was observed that unmethylated alleles were not amplified during PCR. It was also observed that methylated alleles (full mutation, some pre-mutation, and approximately 50% of each allele for females) were resistant to the cutting of the Hha I enzyme and were, therefore, amplified during PCR. For the 158 samples analyzed for the validation of the Hha I digest with Southern blot of the Celera/Abbott Fragile X PCR, 98.0% concordance with the clinical, genomic DNA Southern blot results was observed for the 154 samples with results (FIG. 11). All of the four discrepancies were due to increased sensitivity for size mosaicism using the Celera/Abbott PCR with SB. Overall, the analysis showed 100% sensitivity and 100% specificity for discriminating normal from abnormal samples, when compared to previous laboratory testing results. In addition, the analysis showed 100% sensitivity and 100% specificity for detecting methylation abnormalities. In sum, these results indicate that the methylation state of a Fragile X allele can be determined using pre-PCR digestion of template DNA with the methylation-sensitive restriction endonuclease Hha I.

### Example 7

### Detection of Methylation State with BsaHI

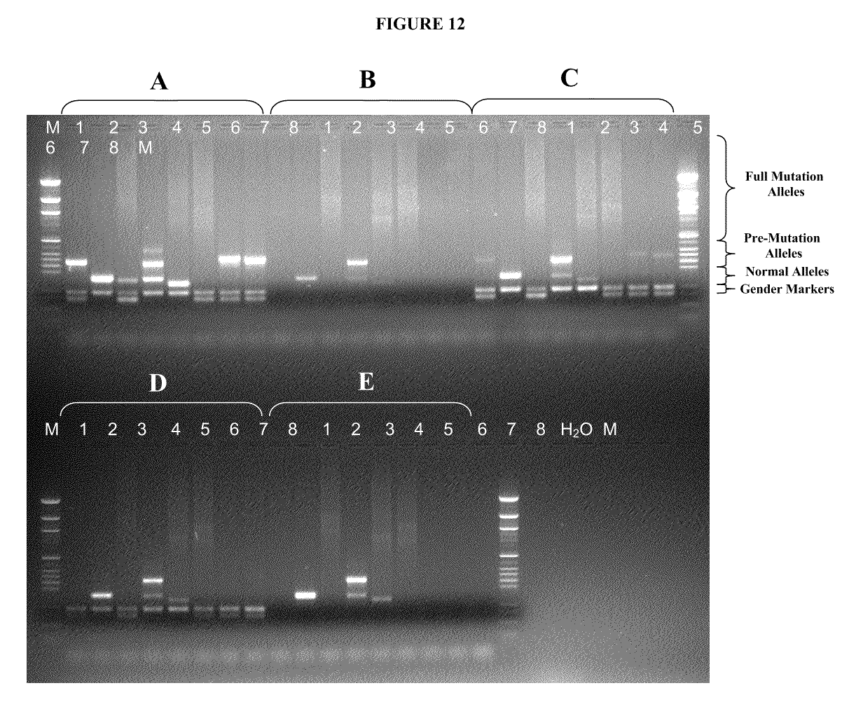

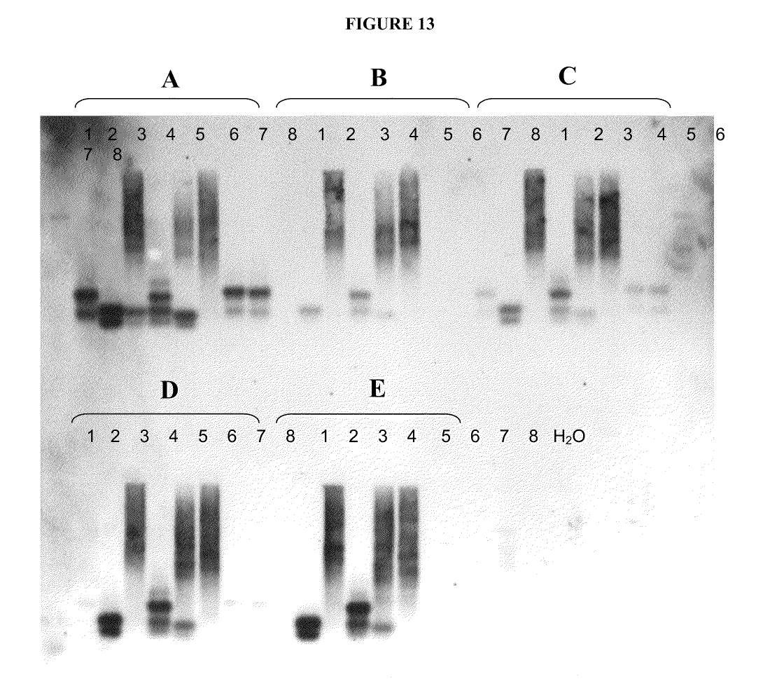

Although pre-digestion of genomic DNA with Hha I enzyme before FX PCR is an effective method for detection of FMR1 methylation state, the method lacks control measures for normal and premutation males, such that no amplification after digestion with Hha I is indistinguishable from a failed amplification. Therefore, a search was performed to identify alternate methylation-sensitive restriction endonucleases with cut sites within the FMR1 targeted region that would differentially digest unmethylated FMR1 alleles, but leave the gender X/Y markers intact. Genomic DNA was digested independently with 3 alternate methylation sensitive enzymes: Sfo I, BsaHI, and BstUI; and then compared to undigested DNA and DNA digested with Hha I (FIG. 12). The restriction endonuclease-treated (and untreated) DNA was amplified using the Fragile X PCR kit (Celera/Abbott). PCR products were size separated on an agarose gel stained with ethidium bromide (FIG. 12). PCR products were transferred to a nylon membrane by Southern blotting (FIG. 13). Of the 3 methylation sensitive enzymes evaluated, BsaHI appeared to give the most distinguishable pattern of expected allele methylation and also left the gender markers intact. BsaHI has one GGCGCC recognition site within the targeted FMR1 region (FIG. 7). BsaHI cleaves double stranded DNA at the GGCGCC recognition sequence only if the cytosine residues are unmethylated.

### Example 8

### Validation of FMR1 Methylation State Detection with BsaHI

Previously examples demonstrated that the digestion of genomic DNA with methylation-sensitive restriction enzymes before the Celera/Abbot Fragile X PCR is effective at determining the methylation status of pre-mutation and full mutation FMR1 alleles. Here we present the validation of one of these enzymes, BsaHI. To validate the methylation state determination process with BsaHI, 79 samples from the sample set used for the validation of the Hha I enzyme for the same process plus an additional 5 prenatal (amniotic fluid) samples and one normal male sample were digested with BsaHI enzyme before amplification using the Celera/Abbott Fragile X PCR kit reagents. Following the PCR amplification, all procedures were performed as described above.

Included in this experiment was a mixture of 23 full mutation carrier females and affected males. Thirty-four pre-mutation and intermediate allele carriers were also included. To check for potential false positives, 14 normal samples previously tested for Fragile X in the laboratory were included. Fourteen samples mosaic for size (combinations of full, pre-mutation, intermediate, and normal alleles) and/or methylation were also assayed. In addition, 10 of the samples were prenatal (amniotic fluid or CVS) and classified within the normal (n=3), pre-mutation/intermediate (n=5), full mutation (n=1) and mosaic (n=2) groups.

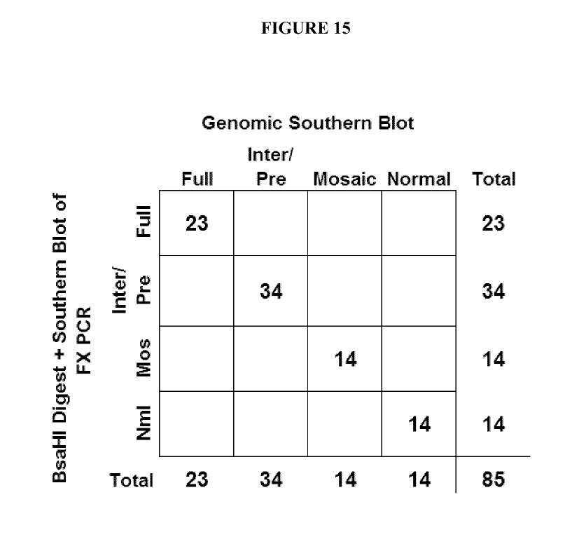

It was observed that unmethylated alleles were not amplified during PCR. It was also observed that methylated alleles (full mutation, some pre-mutation, and approximately 50% of each allele for females) were resistant to the cutting of the BsaHI enzyme and were, therefore, amplified during PCR. For the 85 samples analyzed for the validation of the BsaHI digest with Southern blot of the Celera/Abbott Fragile X PCR, 100.0% concordance with the clinical, genomic DNA Southern blot results was observed (FIG. 15). Overall, the analysis showed 100% sensitivity and 100% specificity for discriminating normal from abnormal alleles with methylation abnormalities, when compared to previous laboratory testing results. In addition, the analysis showed 100% sensitivity and 100% specificity for detecting methylation abnormalities. However, prenatal samples with normal alleles had minor resistance to cutting by the BsaHI enzyme at its recognition site (normal allele was slightly methylated), compared to the Hha I enzyme. This resistance for normal alleles should not impact interpretation of the abnormal alleles, which are the focus of this follow-up analysis. In sum, these results indicate that the methylation state of a Fragile X allele can be determined using pre-PCR digestion of template DNA with the methylation-sensitive restriction endonuclease BsaHI.

### Example 9

### Detecting Fragile X Alleles without Manual Blotting

As described in the previous sections, post-PCR SB can replace genomic DNA SB for amplifying full Fragile X mutations without loss of clinical sensitivity or specificity. Although post-PCR SB is much less labor- and time-intensive than genomic SB, the method is cumbersome relative to most modern PCR protocols. Thus, it would be advantageous to modify the post-PCR SB protocol to eliminate the use of the manual blotting procedure. The post-PCR amplification product is hybridized with a fluorescently labeled probe. The probe, with sequence identical or similar to the post-PCR SB probe described herein, is labeled with a different fluorescent dye than the PCR primers. The resulting labeled PCR product is analyzed by capillary electrophoresis with a laser-induced fluorescence detector (CE-LIF) (Applied Biosystems). Alternatively, the hybridization probe is a standard oligonucleotide or a peptide nucleic acid (PNA). When a PNA probe is used, excess probe is not be expected to electrokinetically inject onto the CE-LIF column. Thus, there is no need to remove unhybridized probe.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing Fragile X alleles in a mammal, said method comprising
(a) amplifying nucleic acid obtained from said mammal to obtain amplified nucleic acid, wherein said amplified nucleic acid comprises a sequence from a regulatory region for a Fragile X polypeptide-encoding sequence, wherein said sequence comprises a CGG location flanked by a 3′ non-CGG sequence and a 5′ non-CGG sequence;
(b) detecting the size of said amplified nucleic acid using a Southern blot and a oligonucleotide probe, wherein said oligonucleotide probe hybridizes to a repeated CGG sequence of said amplified nucleic acid, wherein the presence of a size diagnostic of greater than 200 CGG repeats at said CGG location in said amplified product indicates that said mammal comprises a Fragile X full mutant allele, wherein the presence of a size diagnostic of 59 to 199 CGG repeats at said CGG location in said amplified product indicates that said mammal comprises a Fragile X pre-mutation allele, wherein the presence of a size diagnostic of 45 to 58 CGG repeats at said CGG location in said amplified product indicates that said mammal comprises a Fragile X intermediate mutant allele, and wherein the presence of a size diagnostic of less than 45 CGG repeats at said CGG location in said amplified product indicates that said mammal comprises a non-mutant Fragile X allele.

2. The method of claim 1, wherein said mammal is a human.

3. The method of claim 1, wherein said nucleic acid is genomic DNA.

4. The method of claim 1, wherein said method comprises classifying said mammal as having a Fragile X full mutant allele, a Fragile X pre-mutation allele, a Fragile X intermediate mutant allele, or a non-mutant Fragile X allele.

5. The method of claim 1, wherein said method comprises assessing a methylation state of said regulatory region for a Fragile X polypeptide-encoding sequence by digesting said nucleic acid with a methylation-sensitive restriction enzyme prior to said amplifying step, wherein said amplifying step occurs when said nucleic acid is methylated at a recognition site for said methylation-sensitive restriction enzyme.

6. A method for assessing Fragile X alleles in a mammal, said method comprising
(a) amplifying nucleic acid obtained from said mammal to obtain amplified nucleic acid, wherein said amplified nucleic acid comprises a sequence from a regulatory region for a Fragile X polypeptide-encoding sequence, wherein said sequence comprises a CGG location flanked by a 3′ non-CGG sequence and a 5′ non-CGG sequence, and wherein said amplified nucleic acid is labeled with a nucleic acid label; and
(b) detecting the size of said amplified nucleic acid using capillary electrophoresis.

7. The method of claim 6, wherein said nucleic acid label is a fluorescent label.

8. The method of claim 6, wherein said capillary electrophoresis comprises laser-induced fluorescent detection.

9. The method of claim 8, wherein said fluorescent detection is performed using an Applied Biosystems ABI 3730x1 capillary electrophoresis instrument.


SEQUENCE LISTING (6 sequences)

SEQ ID NO: 1 - DNA, 301 bp, Homo sapiens
tacacccgcagcgggccgggggttcggcctcagtcaggccgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagct

SEQ ID NO: 2 - DNA, 21 bp, Artificial Sequence
  Synthetic Oligonucleotide
gacggaggcgccgctgccagg

SEQ ID NO: 3 - DNA, 21 bp, Artificial Sequence
  Synthetic Oligonucleotide
gtgggctgcgggcgctcgagg

SEQ ID NO: 4 - DNA, 21 bp, Artificial Sequence
  Synthetic Oligonucleotide
ccgccgccgccgccgccgccg

SEQ ID NO: 5 - DNA, 21 bp, Artificial Sequence
  Synthetic Oligonucleotide
tcaggcgctcagctccgtttc

SEQ ID NO: 6 - DNA, 23 bp, Artificial Sequence
  Synthetic Oligonucleotide
cctccatcttctcttcagccctg